(19) United States
(12) Patent Application Publication
(10) Pub. No.: US 2004/0219652 A1
(43) Pub. Date: Nov. 4, 2004

(54) Removing deposits of animal dung

(76) Inventors: Anthony Dale Covington, Northampton (GB); Christine Stella Evans, London (GB); Millie Ullah, London (GB)

Correspondence Address: FISH & RICHARDSON PC, 225 FRANKLIN ST, BOSTON, MA, 02110, US

(21) Appl. No.: 10/855,250

(22) Filed: May 27, 2004

Related U.S. Application Data
(63) Continuation-in-part of application No. PCT/GB03/01113, filed on Mar. 18, 2003.
(63) Continuation-in-part of application No. PCT/GB03/03384, filed on Aug. 4, 2003.

(30) Foreign Application Priority Data
Mar. 19, 2002 (GB) 0206464.0
Aug. 2, 2002 (GB) 0218001.6

(51) Int. Cl. C12N 9/00

(52) U.S. Cl. 435/183

(57) ABSTRACT

An enzyme mixture, suitable for removal of dung from animal skins and farm buildings, is prepared by cultivating white rot fungi under conditions that induce formation of a significant amount of laccase, and provide a mixture of the three enzymes cellulase, xylanase and laccase (ligninase).

### RELATED APPLICATIONS

This application is a continuation-in-part, and claims the benefit under 35 USC § 120, of both International application PCT/GB03/01113, filed Mar. 18, 2003, and International application PCT/GB03/03384, filed Aug. 4, 2003.

### BACKGROUND

This invention is concerned with cleaning dung deposits from animal skins and other locations by providing a composition for degrading dung deposits on animal hides and skins used for leather manufacture, and in livestock handling areas. This typically involves cattle, sheep and pig skins, but the problem of coated dung is especially prominent on skins from cattle. In particular, the present invention is concerned with the production of enzymes in a mixture, specifically designed to degrade the dung.

Dung on cattle creates problems for hygiene on the dairy farm and more particularly at the abattoir, where there is risk of contaminating the carcase with faecal organisms, notably including E. coli O157. Typically this is not addressed by the respective industries, creating a residual problem that must be addressed by the global leather industry, particularly in respect of beef cattle which form the biggest source of hides for the leather industry.

Dung must be removed from the hides in the early stages of processing, as part of the cleansing operations, leading to tanning and the production of high quality leather.

Removal of dung is difficult; the composite material created by hair and dry dung is resistant to solubilisation, even in the presence of surfactants. It is accepted in the industry that even the enzymes offered as soaking auxiliaries do not have any useful effect in this regard: those enzymes include proteases, lipases and amylases, but no claims are made by the supply houses for any positive effect on dung. In studies of the effects of enzymes on the solubilisation of dry dung, it was confirmed that those types of soaking enzymes are ineffective (Enzymatic removal of dung from hides. N. Auer, A. D. Covington, C. S. Evans, M. Natt, M. Tozan; J. Soc. Leather Technol. Chem., 83(4),215, 1999.). Therefore, tanners are obliged to risk bacterial damage in prolonged soaking, or to remove the dung with hair, incurring additional chemical cost and limiting the options for disposing of the contaminated hair.

In GB 2,325,241 it is demonstrated that dung is removed efficiently and effectively by targeting the main components of the dung with specifically acting enzymes: the lignocellulosic material, from degraded plant cell walls, can be solubilised with a mixture of cellulase, and xylanase, optionally ligninase.

Subsequent studies by the present inventors have showed that the lignocellulosic material is solubilised as the complex, rather than by selective damage of one or two of the constituents. This indicates that it is necessary to degrade the lignin which surrounds the fibres to expose the cellulose to attack, and at the same time to break the hemicellulose linkages between the cellulose chains, in order to dissolve the complex.

### SUMMARY OF THE INVENTION

Accordingly, the present inventors' solution to the problem of dung in the tannery is to apply a treatment of the three enzymes, at an optimum activity ratio. However, although cellulase and xylanase are available in commercial quantities, there is at present no commercial source of ligninase. Therefore, there is a need to produce the ligninase by large scale fermentation of a suitable microorganism. In this context the present inventors have sought to create the conditions which would force a microorganism to express the mixture of enzymes required for dung removal.

This invention is based on the finding that white rot fungi can be induced to produce a mixture of enzymes suitable for removal of dung from animal skins as a preparative step in production of leather.

In one aspect the present invention provides an enzyme mixture suitable for degrading dung deposits to assist in cleaning dung deposits from animal skins and other locations, which mixture has been prepared by cultivating a fungus selected from the class of White Rot Fungi in a liquid growth medium, and harvesting the enzymes produced by the fungus from the liquid growth medium.

A useful blend of the enzymes cellulase, xylanase and laccase (ligninase) is obtained by carrying out the cultivation in the presence of a suitable inducer, which most preferably is dung or a liquid dung extract, especially an aqueous extract of cattle dung.

Accordingly in another aspect the present invention provides a method of preparing an enzyme mixture suitable for degrading dung deposits on animal skins and other locations, which comprises cultivating a fungus selected from the class of White Rot Fungi in a liquid growth medium in the presence of dung or a liquid dung extract, especially an aqueous extract of cattle dung, as an inducer, and harvesting the enzymes produced by the fungus from the liquid growth medium.

The principle aim of the present invention is to produce a mixture of the three enzymes cellulase, xylanase and laccase (ligninase) in a blend in which the amounts of the enzymes are suitably proportioned to be effective for degrading dung deposits so that they can be easily removed from the surface on which they are deposited.

Suitable white rot fungi are found (but not exclusively) in the family Polyporaceae. Especially suitable are fungi of the species Coriolus, Pleurotus, and Ganoderma, in particular Coriolus versicolor (also known as Trametes versicolor), Pleurotus ostreatus and Ganoderma applanatum. Other suitable white rot fungi can easily be determined by routine testing for rate of growth, ability to produce all three enzymes, levels of enzyme activities etc . . .

Some white rot fungi decompose lignin by production of a peroxidase, which requires hydrogen peroxide as a cofactor, rather than laccase. A typical example is the species Phanerochaete, especially Phanerochaete chrysosporium. These white rot fungi are within the scope of the present invention, but the resultant enzyme mixtures are less preferable for the treatment of animal skins because of the need to provide a source of hydrogen peroxide for the peroxidase to act.

The present inventors have found that white rot fungi that produce a mixture of cellulase, xylanase and laccase often do not produce laccase in sufficient quantities for optimum treatment of animal skins to remove dung deposits. However it has been discovered that this problem can be overcome by cultivating the fungus in the presence of a suitable inducer. Advantageously the inducer to promote production of the desired blend of enzymes to remove dung deposits is cattle dung, preferably in sterile form, as a powder or liquid extract, especially an aqueous extract. Handling of the inducer and the accuracy of measurement is improved by use of a liquid extract of dung as the inducer.

In tests carried out by the present inventors on the fungi Coriolus versicolor, Pleurotus ostreatus and Ganoderma applanatum, Coriolus versicolor and Pleurotus ostreatus were the fastest growing species, covering a 7 cm malt-agar Petri plate with hyphae from a central inoculum within six days, whereas Ganoderma applanatum, took twelve days.

C. versicolor and P. ostreatus produced similar amounts of cellulase and xylanase in the liquid media with cellulose or xylan as substrates over a ten day growth period, but differed in their production of laccase. P. ostreatus produced only low levels of laccase over ten days, with most laccase produced after growing for twenty days or more, when cellulase and xylanase activities had diminished considerably. Laccase activity was not increased significantly in the presence of a lignin mimic inducer in the first ten days of culture. In contrast, laccase production by C. versicolor doubled in the presence of an inducer compound, with the highest amount of laccase produced by any organism after eight days growth.

Surprisingly, it was found that the ratios of the three enzyme activities required to treat dung could be controlled by the nature of the growing medium, in particular, the difficulty of producing enough ligninase (laccase) could be overcome by adding a growth medium auxiliary as an inducer. Thus, the required enzyme mixture can be produced in a single fermentation step.

Unexpectedly, it was found that the inclusion of dung in the cultivation process significantly broadened the peak of laccase production. The dung may be used either dried and preferably ground, and preferably sterilised before use; or as a liquid, preferably aqueous, dung extract. This is of great value in the context of commercial production, since it greatly assists in the ability to harvest a suitably proportioned enzyme mixture.

In the present invention, the fungi are cultivated in a liquid nutrient medium with a nitrogen source and a carbon source, and preferably an inducer in the form of sterile dung or dung extract. After a suitable period of growth, fungal growth is removed and enzymes in the culture fluid are harvested.

Suitably the fungi are added to the nutrient medium in pelletised form, to assist in subsequent removal by filtration, together with any dung residue remaining if powdered dung is used as inducer. The filtrate containing the enzymes is preferably concentrated, for example using a membrane concentrator with a cut off at 10,000 Daltons. Then the concentrate is preferably dried. Freeze drying will provide the desired enzymes as a lyophilised powder. Spray drying or other drying systems may also be used.

The powder may be stored or packaged for future use as a cleaning composition, for example in leather preparation, to remove dung from animal skins as proposed in GB 2,325,241, the entire disclosure of which is incorporated herein by reference. Suitably, before use as a cleaning aid, the enzyme powder is mixed with an inert bulking agent, so that technicians are able weigh out enzyme dosages in, for example 10 or 100 gm units rather than as single figure gram units. Alternatively the enzyme mixture, or bulked mixture, may be pre-packaged in unit doses. The bulking agent is preferably selected so that it will not leave a residue on the treated skins or other cleaned surfaces, or affect treatment pH. Sodium chloride may be used.

Alternatively, a lyophilised powder may be reconstituted with water, to provide the user with a liquid concentrate.

Whether supplied to cleaning personnel in either powder or liquid form, the enzyme composition is preferably applied to the dung deposit as an aqueous solution or dispersion, optionally formulated with thickening agents to prevent unnecessary spread of the formulation, or with surfactants to assist in the cleaning process, and/or with stabilisers.

Using the above process with an inducer, enzyme mixtures that are roughly in the proportion of 200 units cellulase, 250 units of xylanase, and 40 units of laccase can be obtained. This provides an effective cleaning composition as shown in the Examples below. In practice effective cleaning can be achieved provided that cellulase and xylanase are present at levels of at least about 150 units each Laccase can be present in much smaller amounts, for example 10 units or more.

If the fermentation to produce the enzyme mixture is carried out at or close to the tannery, then it may be suitable or appropriate to use the enzyme mixture in leather processing as the liquid concentrate obtained after harvesting the enzymes by removing small molecules. Alternatively, the lyophilised powder may be reconstituted with water, to add the enzymes to the treatment bath as a liquid

Applying the teaching of this invention has two aspects: first the choice of microorganism(s) from the indicated white rot fungi, which determines the types of enzymes capable of being expressed and second the nature of the growing medium, which controls the amounts of the enzymes. The latter depends to a large degree on the choice of auxiliary/inducer, to produce the required enzyme mixture to treat a particular type of animal faeces: most suitably this will be the faeces itself, especially cattle dung.

The basis of the invention is the means of producing the desired enzymes in suitable ratios for cleaning skins in a single fermentation. In the preparation of leather, the ability to remove the dung in the first soak, the dirtiest step, enables the pollution to be confined to a small volume for effluent treatment. Moreover, efficient early cleaning of the hide can open up new processing options for the leather industry, offering better leather quality and reduced environmental impact. In particular the simple change to removing the flesh after soaking is a highly desirable alternative to fleshing part processed hide, when it is alkaline with hair removed and thereby difficult to handle.

A further use of the enzyme mixtures obtainable by this invention is to clean the skins and pelts of live animals before slaughter, to improve hygiene in the abattoir. Another use is the removal of dung deposits in and around farm-buildings, to improve hygiene in the milking parlour and in animal accommodation areas.

The invention is further illustrated by the following Examples and by reference to the accompanying drawings, in which:

### DESCRIPTION OF THE DRAWINGS

FIGS. 1a, 1b and 1c show production of enzymes after adding 1% (w/v) inducer after 3, 6, 9 or 12 days into cultures of C. versicolor containing 2% (w/v) carboxymethyl cellulose (CMC) as carbon source;

FIGS. 2a, 2b and 2c shows a comparison of shaker speeds in enzyme production;

FIGS. 3a, 3b and 3c show further results from fermentation of C. versicolor on the 2 litre scale;

FIGS. 4a and 4b show the effects of the cleaning process of the invention on a concrete wall before treatment (4a) and after treatment (4b);

FIGS. 4c and 4d show the effects of the cleaning process of the invention on a painted door before treatment (4c) and after treatment (4d);

FIGS. 5a and 5b show the effects of the cleaning process of the invention on cattle before treatment (5a) and after treatment (5b);

FIGS. 5c and 5d show the effects of the cleaning process of the invention on lambs before treatment (5c) and after treatment (5d).

### DETAILED DESCRIPTION

### EXAMPLE 1

Induced Enzyme Production by C. versicolor in Shake Flask Cultures.

The liquid growth media in these trials were based on a mineral salts medium with ammonium nitrate as nitrogen source (see—E. Abrams; National Bureau of Standards Misc. Publications no. 188. U.S. Dept. of Commerce, Washington) and included carboxymethyl cellulose (CMC) as carbon source. The inventors' proposed inducer for laccase was cow dung collected from a field grazed by cattle.

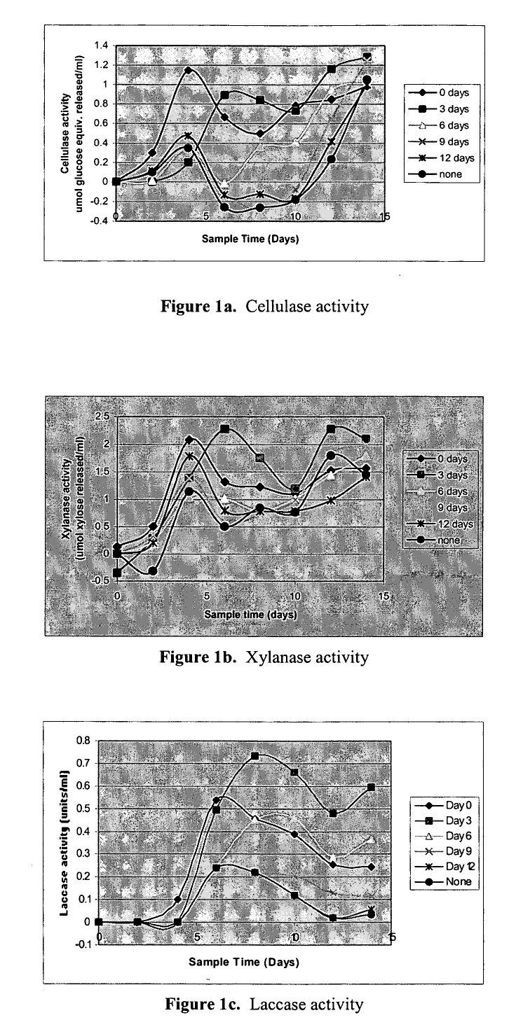

Dried and sterilised cow dung, was added at different time intervals during growth of the fungus in the growth medium. The results are shown graphically in the appended FIGS. 1a, 1b and 1c which show the effects on the production of enzymes of adding 1% (w/v) inducer after 3, 6, 9 or 12 days into cultures of C. versicolor containing 2% (w/v) CMC as carbon source.

For laccase production, addition of the inducer after three days of fungal growth gave the highest yield of laccase in cultures at day 8 of growth. For cellulase and xylanase activities, addition of the inducer after three days also yielded the highest enzymes activities, but from day 6 onwards.

TABLE I
The effect of inducer on the production of laccase.

| | Inducer offered (% w/v) | | | | |
|---|---|---|---|---|---|
| | 0 | 0.1 | 0.2 | 0.5 | 1.0 |
| Laccase activity at day 8 | 0.2 | 0.22 | 0.37 | 0.62 | 0.59 |
| (ΔOD₄₄₀/ml/min) | | | | | |

### EXAMPLE 2

The Role of Carbon Source in Stimulated Enzyme Production from C. versicolor in Shake Flask Cultures.

The effects of different carbon sources on enzyme production were investigated: using glucose, crystalline cellulose powder or CMC, each at 2% w/v in the medium. The results are shown in Table II.

TABLE II
The effect of carbon source and inducer on the activities of
enzymes cultured from C. versicolor.

| | Enzyme activities | | |
|---|---|---|---|
| | | Xylanase (μmol | Laccase |
| | Cellulase (μmol | glucose equivalent | (ΔOD₄₄₀ |
| Media | glucose released | released per ml | per ml |
| composition | per ml per h) | per h) | per min) |
| 2% CMC | 0.72 | 1.02 | 0.20 |
| 2% CMC + | 1.58 | 1.57 | 0.62 |
| 1% inducer | | | |
| 2% cellulose | 0.80 | 1.22 | 0.22 |
| 2% cellulose + | 0.95 | 1.21 | 0.32 |
| 1% inducer | | | |
| 2% CMC + | 1.64 | 0.95 | 0.78 |
| 0.5% glucose + | | | |
| 1% inducer | | | |
| 2% CMC + | 1.53 | 0.89 | 1.8 |
| 1% glucose + | | | |
| 1% inducer | | | |

From these tests, CMC is the preferred carbon source for production of all three enzymes. Addition of 0.5-1.0% inducer at day 3 of growth stimulated enzyme production by 80-100% from day 6 for xylanase and laccase and from day 8 for cellulase. Addition of different concentrations of CMC was investigated (0.5 to 2%) for the effect on enzyme production: all enzyme activities increased as the concentration of CMC was increased in the medium, up to 2% CMC.

The optimum conditions for simultaneous production of cellulase, xylanase and laccase activities in shake flasks were 2% w/v CMC in the medium as carbon source, 0.5-1.0% w/v inducer added at day 3 of growth. The cultures reached the optimum enzyme activities at 8 days growth, with approximately equal cellulase and xylanase activities. For application to treating dirty cattle hides, GB 2,325,241 indicates the preferred ratio of cellulase to xylanase as 2:1, together with sufficient laccase activity.

If 1% glucose was added to the CMC medium, the titre for laccase activity was increased substantially, three fold, with cellulase unchanged, but xylanase titre reduced by 30%, see Table IV. This resulted in a cellulase to xylanase ratio closer to 2:1, but with substantially increased laccase activity, which meets the preferred mixture requirements more closely.

### EXAMPLE 3

The Effect of Agitation on Shake Flask Cultures of C. versicolor.

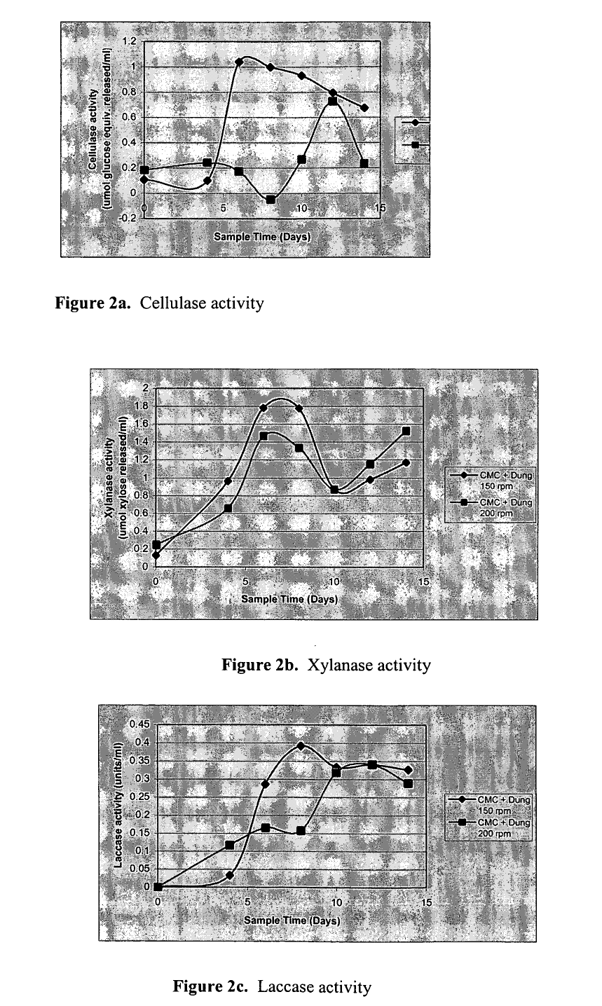

The agitation rate of the cultures (affecting availability of dissolved oxygen) was found to be critical in maximising enzyme production. At day 8 of growth, with the inducer added at day 3, cellulase, xylanase and laccase activities maintained higher levels when agitation was at 150 rpm, compared with 200 rpm. Optimum activities of all three enzymes occurred at day 8 under these conditions, as shown in FIGS. 2a, 2b and 2c comparing shaker speeds in enzyme production.

### EXAMPLE 4

Simultaneous Production of Cellulase, Xylanase and Laccase by Culturing C. versicolor in 2 Litre Bioreactors.

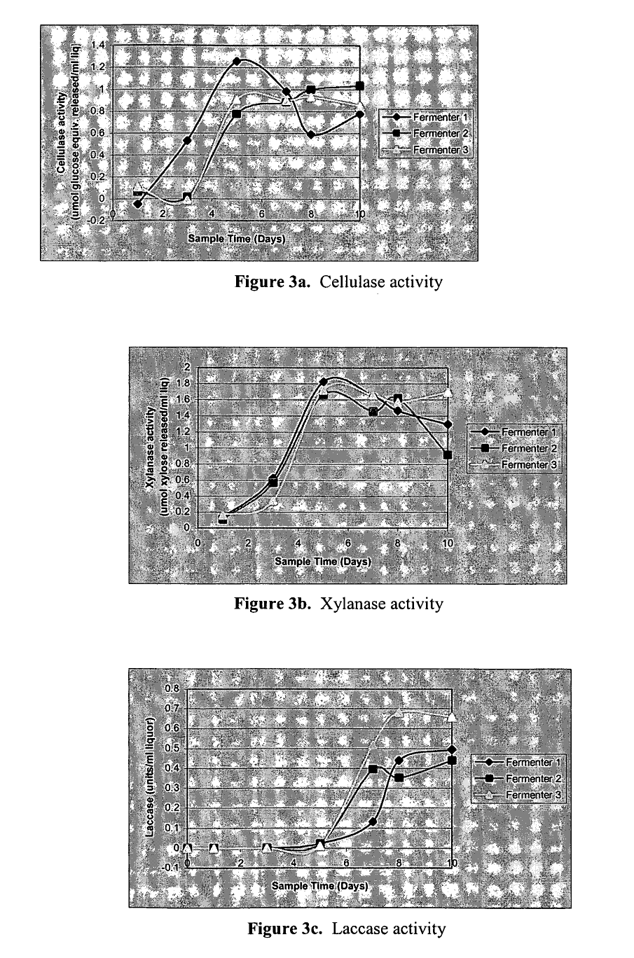

The medium optimised for shake flasks, using 2% CMC as carbon source and addition of inducer on day 3 of fungal growth, was used in the bioreactor, with agitation at 150 rpm. FIGS. 3a, 3b and 3c show enzyme activities from typical fermentation, with maximum activities occurring from day 5 to day 10 of growth for cellulase and xylanase and at day 8 for laccase. Dissolved oxygen concentration was maintained between 20 and 100% throughout the fermentation,

### EXAMPLE 5

Simultaneous Production of Cellulase, Xylanase and Laccase by Culturing C. versicolor in 20 Litre and 75 Litre Bioreactors.

The same conditions as described for a 2 litre bioreactor, given in Example 4, were used for growing the fungus in a 20 litre bioreactor. Dissolved oxygen concentration was maintained at 40 to 100%. It was observed that maximum cellulase, xylanase and laccase production was obtained between days 6 and 12 of growth.

Using a 75 litre bioreactor, similar enzyme titres were obtained, providing the dissolved oxygen concentration did not fall below 30%.

### EXAMPLE 6

Stability of the Enzyme Mixture

Enzymes in the culture fluid, harvested on day 8 of growth, were concentrated through a membrane concentrator with a cut off at 10,000 Daltons, then the concentrate was freeze dried.

The powder was stored at room temperature, 4° C. and −20° C. and the activity was assayed over a three month period. Laccase activity disappeared after 3 months at room temperature and reduced by 50% at −20° C. Cellulase and xylanase activities had not decreased after three months at −20° C. or 4° C., but a slight reduction was observed in xylanase activity after storage at room temperature.

### EXAMPLE 7

Preparation of Dung-Extract for use as Inducer

The procedures described in the preceding Examples use dried sterilised cow dung as inducer. Handling of the inducer and the accuracy of measurement is improved by use of a liquid extract of dung as the inducer. An aqueous extract is prepared as follows.

To two parts by volume of dung is added one part by volume of water. The mixture is heated to 70° C., under constant mixing, and then held at 70° C. for 1 hour under constant mixing. After cooling, the mixture is filtered through muslin, allowing tiny particles to pass through. The thus obtained liquid dung extract is sterilised by autoclaving at 121° C. for 30 minutes, allowed to cool and stored at room temperature until required.

### EXAMPLE 8

Removing Dung from Walls of Dairy Parlours and Farm Buildings.

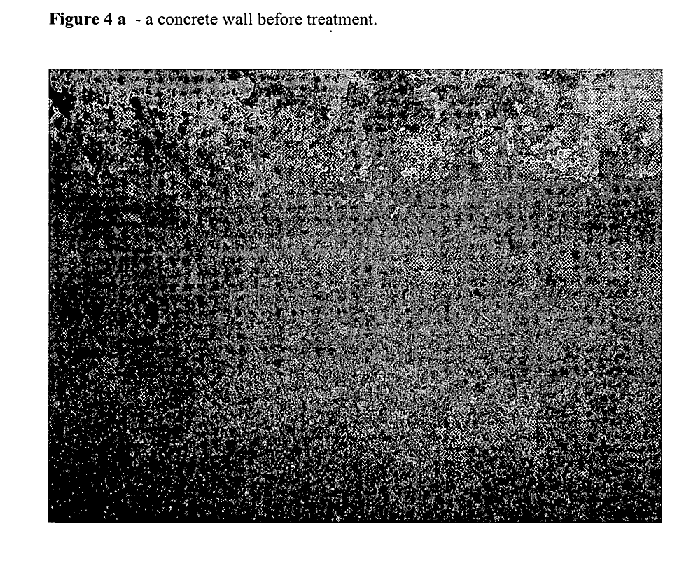

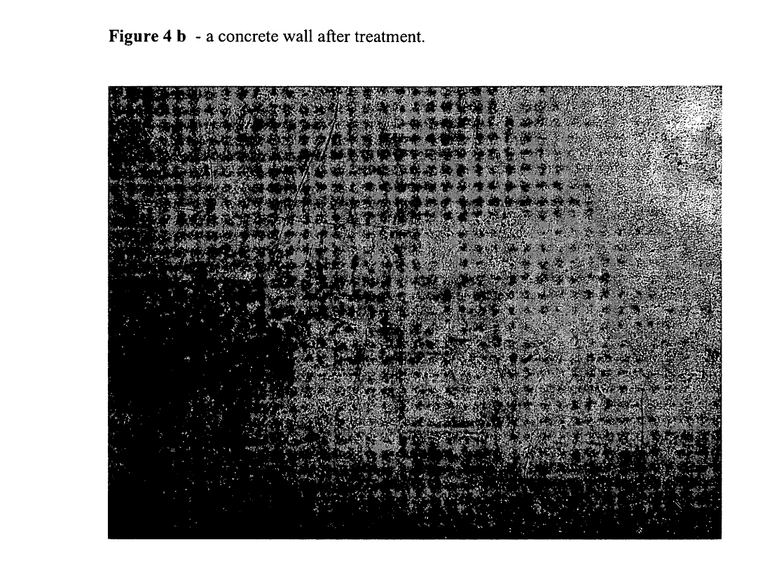

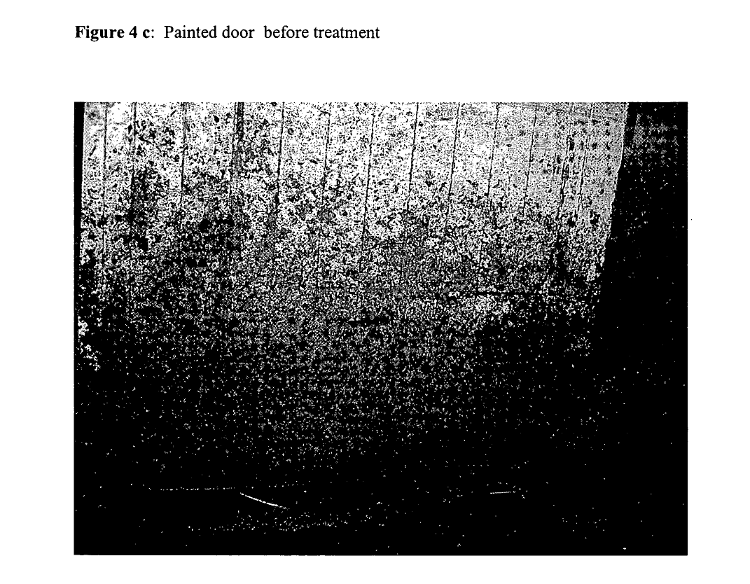

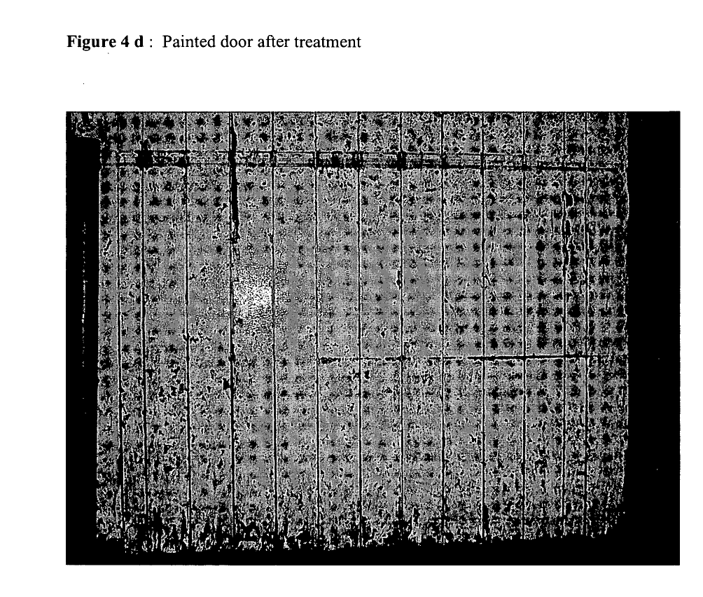

Dung-clad walls in a dairy parlour were sprayed with water to moisten the dung, then sprayed with an aqueous solution of a crude mixture of the enzymes, cellulase, xylanase and laccase, obtained by cultivation as in Example 5. After 60 min the walls were resprayed with water from a medium pressure hose, which removed the softened and degraded dung. The effect of the enzyme mixture is demonstrated in FIGS. 4a and 4b which show respectively photographs of a concrete wall before and after treatment.

Dung-spattered doors of farm buildings were similarly treated. The effect of the enzyme mixture is demonstrated in FIGS. 4c and 4d which show respectively photographs of a painted door before and after treatment.

### EXAMPLE 9

Removing Dung from the Hides of Live Sheep and Cattle.

Live animal trials were carried out using an enzyme mixture obtained by cultivation as in Example 5, in which the approximate amounts of the enzymes used were 200 units cellulase, 250 units of xylanase, and 40 units of laccase.

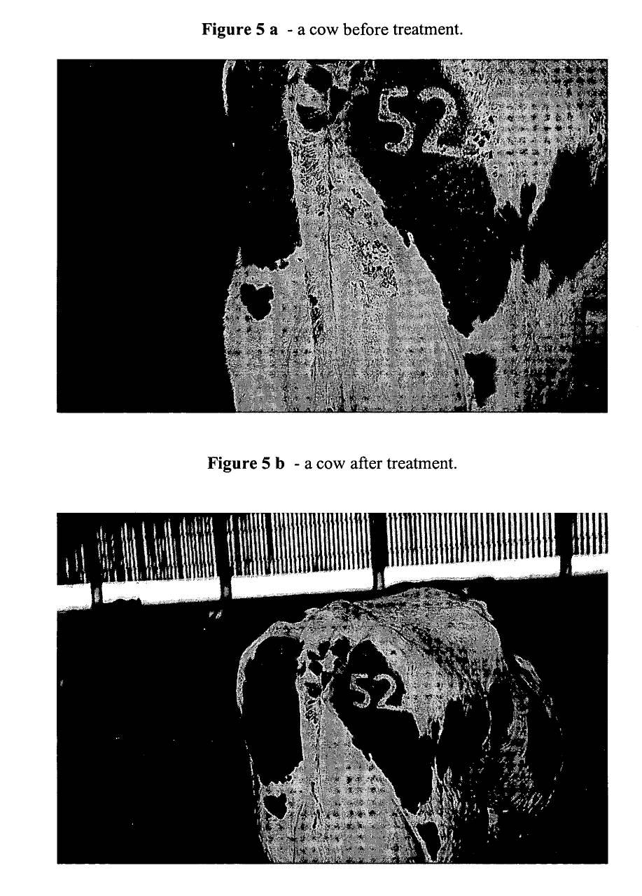

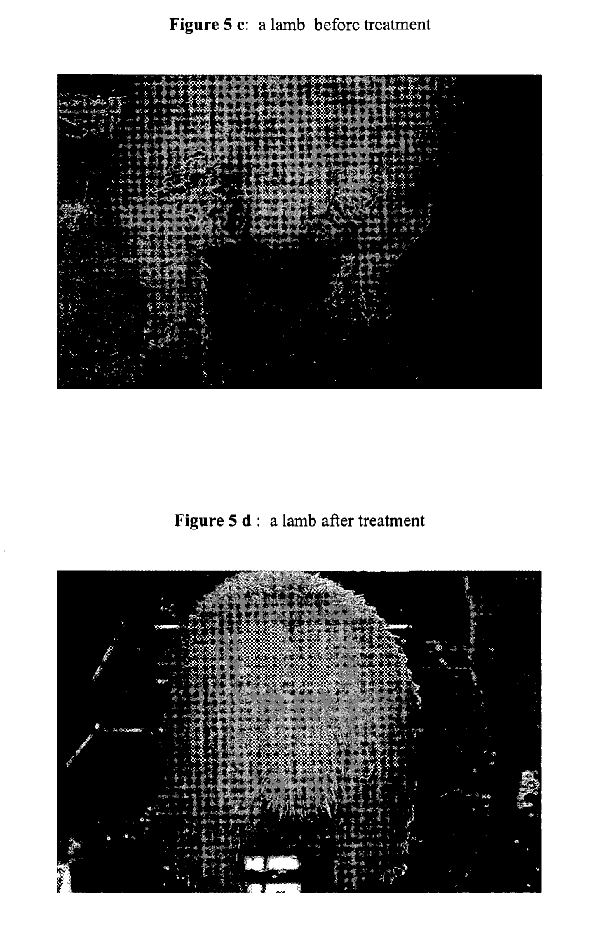

Cattle were sprayed on dung-affected areas with an enzyme solution of 500 mg of enzyme mixture in 500 ml water. After 60 min the dung was removed by spraying with water. FIGS. 5a and 5b respectively are photographs of the hind-quarters of a cow, before and after treatment.

Lambs were sprayed on dung-affected areas with a solution of 400 mg of the enzyme mixture in 400 ml water. After 60 min the dung was removed using a water spray and a soft brush. FIGS. 5a and 5b respectively are photographs of the hind-quarters of a lamb, before and after treatment.

### OTHER EMBODIMENTS

The specific examples above are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What we claim is:

1. An enzyme composition containing the enzymes cellulase, xylanase and laccase in proportions suitable for degrading dung deposits to assist cleaning, which composition has been obtained by cultivating a fungus selected from the class of White Rot Fungi in a liquid growth medium, and harvesting the enzymes produced by the fungus from the liquid growth medium.

2. A composition as claimed in claim 1 in which the fungus is cultivated in the presence of an inducer which increases the production of laccase.

3. A composition as claimed in claim 2 in which animal dung is used as inducer.

4. A composition as claimed in claim 3 in which the dung is used as dried powdered dung.

5. A composition as claimed in claim 3 in which the dung is used as a liquid dung extract.

6. A composition as claimed in claim 5 in which the liquid extract is an aqueous extract.

7. A composition according to claim 1 in which the fungus is selected from the group consisting of the species Coriolus, Pleurotus and Ganoderma.

8. A composition according to claim 7 in which the fungus is selected from the group consisting of Coriolus versicolor, Pleurotus ostreatus and Ganoderma applanatum.

9. A composition according to claim 3 in which the fungus has been cultivated in the presence of cattle dung.

10. A composition according to claim 1 in which the enzymes are present as a lyophilised powder.

11. A composition according to claim 10 further comprising an inert bulking agent.

12. A composition according to claim 1 in which the enzymes are present as in an aqueous solution.

13. A method of cleaning dung from the skin and pelts of live animals which comprises treating said animals externally with an aqueous solution of an enzyme composition according to claim 1.

14. A method of cleaning dung from animal skins before tanning to produce leather which comprises treating said skins with an aqueous solution of an enzyme composition according to claim 1.

15. A method of cleaning dung deposits from livestock handling areas which comprises treating said deposits with an aqueous solution of an enzyme composition according to claim 1.

16. A method of preparing an enzyme mixture comprising cellulase, xylanase and laccase in proportions suitable for degrading dung deposits on animal skins and other locations, which comprises cultivating a fungus selected from the class of White Rot Fungi in a liquid growth medium in the presence an inducer which increases the production of laccase, and harvesting the enzymes produced by the fungus from the liquid growth medium.

17. A method as claimed in claim 16 in which animal dung is used as inducer.

18. A method as claimed in claim 17 in which the dung is used as dried powdered dung.

19. A method as claimed in claim 17 in which the dung is used as a liquid dung extract.

20. A method as claimed in claim 17 in which the dung is used as aqueous dung extract.

21. A method as claimed in claim 16 in which cattle dung is used as inducer.
